(19) United States
(12) Patent Application Publication
(10) Pub. No.: US 2010/0068262 A1
(43) Pub. Date: Mar. 18, 2010

(54) Method and Compositions for Stimulation of an Immune Response to PSMA using a Xenogeneic PSMA Antigen

(75) Inventors: Alan N. Houghton, New York, NY (US); Clarissa Naftzger, Belmont, CA (US); Setaluri Vijayasaradhi, Madison, WI (US)

Correspondence Address: Larson & Anderson, LLC;re: MSK, P. O. BOX 4928, DILLON, CO, 80435-4928, US

(73) Assignee: SLOAN-KETTERING INSTITUTE FOR CANCER RESEARCH, New York, NY (US)

(21) Appl. No.: 12/622,096

(22) Filed: Nov. 19, 2009

Related U.S. Application Data
(63) Continuation-in-part of application No. 10/285,874, filed on Oct. 31, 2002.
Application No. 10/285,874 is a continuation-in-part of application No. 09/627,694, filed on Jul. 28, 2000, now abandoned.
Application No. 09/627,694 is a continuation-in-part of application No. 09/308,697, filed on May 21, 1999, now Pat. No. 6,328,969.
(60) Provisional application No. 60/036,419, filed on Feb. 18, 1997.

(51) Int. Cl. A61K 9/127; A61K 39/00; A61K 31/7052; A61P 37/04

(52) U.S. Cl. 424/450; 424/184.1; 514/44 R

(57) ABSTRACT

Tolerance of the immune system for endogenous PSMA can be overcome and an immune response stimulated by administration of xenogeneic or xenoexpressed PSMA antigen. For example, mouse PSMA, or antigenically-effective portions thereof, can be used to stimulate an immune response to the corresponding differentiation antigen in a human subject. Administration of xenogeneic antigens in accordance with the invention results in an effective immunity against PSMA expressed by the cancer in the treated individual, thus providing a therapeutic approach to the treatment of cancers expressing PSMA, such as prostate cancer.

This application is a continuation-in-part of U.S. patent application Ser. No. 10/285,874, which is continuation-in-part of U.S. patent application Ser. No. 09/627,694, filed Jul. 28, 2000, which is continuation-in-part of U.S. patent application Ser. No. 09/308,697, filed May 21, 1999, which is a §371 National Phase of International Application No. PCT/US97/22669 filed Dec. 10, 1997. The application also claims benefit under 35 USC §119(e) of U.S. Provisional Application No. 60/036,419 filed Feb. 18, 1997. All of the aforementioned applications are incorporated herein reference.

### FIELD OF THE INVENTION

This application relates to a method and compositions for stimulation of an immune response to PSMA.

### BACKGROUND OF THE INVENTION

Most tumor immunity is mediated by recognition of self-antigens, antigens present in cancer cells that are also found in normal host tissue. Houghton, A. N., J. Exp. Med. 180: 1-4 (1994). This type of immunity is more akin to autoimmunity than to immunity in infectious diseases, where the response is directed at a truly foreign antigen, present in the pathogen but not in host tissue. Evidence of this can be found in the autoimmune sequelae that often follow the development of successful tumor immunity. Bowne, W. B., et al., J. Exp. Med. 190(11):1717-1722 (1999).

Differentiation antigens form one prototype of self-antigens in cancer immunity. Houghton, A. N., et al., J. Exp. Med. 156(6):1755-1766 (1982). Differentiation antigens are tissue-specific antigens that are shared by autologous and some allogeneic tumors of similar derivation, and on normal tissue counterparts at the same stage of differentiation. Differentiation antigens have been shown to be expressed by a variety of tumor types, including melanoma, leukemia, lymphomas, colorectal, carcinoma, breast carcinoma, prostate carcinoma, ovarian carcinoma, pancreas carcinomas, and lung cancers. Typically the expression of these antigens changes as a cell matures and can characterize tumors as more or less differentiated. For example, differentiation antigens expressed by melanoma cells include Melan-A/MART-1, Pmel17, tyrosinase, gp75 and gp100. Differentiation antigens expressed by lymphomas and leukemia include CD19 and CD20/CD20 B lymphocyte differentiation markers. An example of a differentiation antigen expressed by colorectal carcinoma, breast carcinoma, pancreas carcinoma, prostate carcinoma, ovarian carcinoma, and lung carcinoma is the mucin polypeptide muc-1. A differentiation antigen expressed by breast carcinoma is her2/neu. The her2/neu differentiation antigen is also expressed by ovarian carcinoma. Differentiation antigens expressed by prostate carcinoma include prostate specific antigen, prostatic acid phosphatase, and prostate specific membrane antigen (PSMA).

Unfortunately, in most cases, the immune system of the individual is tolerant of these antigens, and fails to mount an effective immune response. For the treatment of cancers where the tumor expresses differentiation antigens therefore, it would be desirable to have a method for stimulating an immune response against the differentiation antigen in vivo. It is an object of the present invention to provide such a method.

### SUMMARY OF THE INVENTION

It has now been found that the tolerance of the immune system for endogenous PSMA can be overcome and an immune response stimulated by administration of xenogeneic PSMA and PSMA (including syngeneic PSMA) expressed in cells of different species. For example, mouse PSMA, or antigenically effective portions thereof, can be used to stimulate an immune response to the corresponding differentiation antigen in a human subject. Administration of xenogeneic or xenoexpressed antigens in accordance with the invention results in an effective immunity against PSMA expressed by the cancer in the treated individual, thus providing a therapeutic approach to the treatment of prostate cancers expressing PSMA.

### BRIEF DESCRIPTION OF THE DRAWINGS

FIG. 1 shows ELISA data comparing mice immunized with immune complexes containing hPSMA, or with DNA vaccines for hPSMA, mPSMA, or empty vector.

FIG. 2 shows immunoblot analysis for recombinant hPSMA (H), mPSMA (M) and human tyrosinase (Ty) stained with anti-TAG and sera from an hPSMA DNA immunized mouse.

FIGS. 3A-D shows flow cytometry data for staining with sera from representative mice used to stain NIH-3T3 cells transduced with empty SFV vector (solid), hPSMA (dark line) or mPSMA (light line).

FIG. 4 shows Western blot analysis of the specificity of representative clones.

FIGS. 5A-E show flow cytometry data for representative hybridoma supernatants isolated from a mouse immunized with hPSMA protein.

FIGS. 6A-B show flow cytometry for cross-reactive hybridomas.

### DETAILED DESCRIPTION OF THE INVENTION

The present invention provides a method for stimulating an immune response to a tissue expressing PSMA in a subject individual. The subject individual is preferably human, although the invention can be applied in veterinary applications to animal species, preferably mammalian (for example horse, dog or cat) or avian species, as well.

As used in the specification and claims of this application, the term “immune response” encompasses both cellular and humoral immune responses. Preferably, the immune response is sufficient to provide immunoprotection against growth of tumors expressing PSMA. The term “stimulate” refers to the initial stimulation of a new immune response or to the enhancement of a pre-existing immune response.

In accordance with the invention, a subject individual is treated to stimulate an immune response to endogenous PSMA by administering a xenogeneic or xenoexpressed PSMA antigen. The term “xenogeneic” denotes the fact that the administered antigen has a sequence peptide different from the PSMA of the species being treated and originates from a different species. For treatments of humans, preferred xenogeneic antigens will be rodent antigens, for example mouse, but could come from other mammals such as dog, cat, cow, or sheep, or from birds, fish, amphibian, reptile, insect or other more distantly related species. The term “xenoexpressed” refers to an antigen which may be syngeneic with the subject individual, but which is expressed in cells of a species different from the subject individual, for example in insect cells.

The term “PSMA antigen” refers to a protein/peptide antigen or to a polynucleotide having a sequence that is expressed in vivo to produce the protein/peptide antigen. In either case, the protein/peptide antigen may be the entire PSMA molecule, or some antigenic portion thereof derived from the extracellular domain. For example, as described below, plasmids were prepared using either full length cDNA or using a truncated portion encoding an amino acid strand.

Administration of a protein/peptide xenogeneic or xenoexpressed PSMA antigen can be accomplished by several routes. First, the xenogeneic PSMA may be administered as part of a vaccine composition which may include one or more adjuvants such as alum, QS21, TITERMAX or its derivatives, incomplete or complete Freund's and related adjuvants, and cytokines such as granulocyte-macrophage colony stimulating factor (GM-CSF), flt-3 ligand, interleukin-2, interleukin-4 and interleukin-12 for increasing the intensity of the immune response. The vaccine composition may be in the form of xenogeneic PSMA antigen in a solution or a suspension, or the PSMA antigen may be introduced in a lipid carrier such as a liposome. Such compositions will generally be administered by subcutaneous, intradermal or intramuscular route.

Vaccine compositions containing protein/peptide xenogeneic or xenoexpressed PSMA antigen are administered in amounts which are effective to stimulate an immune response to the target differentiation antigen in the subject individual. The preferred amount to be administered will depend on the species of the target individual and on the specific antigen, but can be determined through routine preliminary tests in which increasing doses are given and the extent of antibody formation or T cell response is measured by enzyme-linked immunosorbent assay (ELISA) or similar tests. T cell responses may also be measured by cellular immune assays, such as cytokine release assays and proliferation assays.

Xenogeneic PSMA antigen may also be introduced in accordance with the invention using a DNA immunization technique in which DNA encoding the antigen is introduced into the subject such that the antigen is expressed by the subject. Xenogeneic PSMA antigen may also be administered as a purified protein. Proteins can be purified for this purpose from cell lysates using column chromatography procedures. Proteins for this purpose may also be purified from recombinant sources, such as bacterial or yeast clones or mammalian or insect cell lines expressing the desired product.

Xenogeneic PSMA antigen may also be administered indirectly through genetic immunization of the subject with DNA encoding the antigen. cDNA encoding the xenogeneic PSMA antigen is combined with a promoter which is effective for expression of the cDNA in mammalian cells. This can be accomplished by digesting the nucleic acid polymer with a restriction endonuclease and cloning into a plasmid containing a promoter such as the SV40 promoter, the cytomegalovirus (CMV) promoter or the Rous sarcoma virus (RSV) promoter. The resulting construct is then used as a vaccine for genetic immunization. The cDNA can also be cloned into plasmid and viral vectors that are known to transduce mammalian cells. These vectors include retroviral vectors, adenovirus vectors, vaccinia virus vectors, pox virus vectors and adenovirus-associated vectors.

Xenogeneic antigen may also be administered in combination with anti-GITR (glucocorticoid-induced tumor necrosis factor receptor family gene), as described in Cohen A, et al, Agonist Anti-GITR Antibody Enhances Vaccine-Induced CD8+ T-cell Responses and Tumor Immunity, 66 CANCER RES. 4904 (2006).

The nucleic acid constructs containing the promoter, PSMA antigen-coding region and intracellular sorting region can be administered directly or they can be packaged in liposomes or coated onto colloidal gold particles prior to administration. Techniques for packaging DNA vaccines into liposomes are known in the art, for example from Murray, ed., GENE TRANSFER AND EXPRESSION PROTOCOLS, Humana Pres, Clifton, N.J. (1991). Similarly, techniques for coating naked DNA onto gold particles are taught in Yang, Gene transfer into mammalian somatic cells in vivo, CRIT. REV. BIOTECH. 12: 335-356 (1992), and techniques for expression of proteins using viral vectors are found in Adolph, K. ed., VIRAL GENOME METHODS, CRC Press, Florida (1996).

For genetic immunization, the vaccine compositions are preferably administered intradermally, subcutaneously or intramuscularly by injection or by gas driven particle bombardment, and are delivered in an amount effective to stimulate an immune response in the host organism. The compositions may also be administered ex vivo to blood or bone marrow-derived cells (which include APCs) using liposomal transfection, particle bombardment or viral infection (including co-cultivation techniques). The treated cells are then reintroduced back into the subject to be immunized. While it will be understood that the amount of material needed will depend on the immunogenicity of each individual construct and cannot be predicted a priori, the process of determining the appropriate dosage for any given construct is straightforward. Specifically, a series of dosages of increasing size, starting at about 0.1 μg is administered and the resulting immune response is observed, for example by measuring antibody titer using an ELISA assay, detecting CTL response using a chromium release assay or detecting TH (helper T cell) response using a cytokine release assay.

In accordance with a further aspect of the present invention, an immune response against a PSMA antigen can be stimulated by the administration of syngeneic PSMA antigen expressed in cells of a different species, i.e. by xenoexpressed PSMA antigen. In general, the subject being treated will be a human or other mammal. Thus, insect cells are a preferred type of cells for expression of the syngeneic differentiation antigen. Suitable insect cell lines include Sf9 cells and Schneider 2 Drosophila cells. The therapeutic differentiation antigen could also be expressed in bacteria, yeast or mammalian cell lines such as COS or Chinese hamster ovary cells. Host cells which are evolutionarily remote from the subject being treated, e.g. insects, yeast or bacteria for a mammalian subject, may be preferred since they are less likely to process the expressed protein in a manner identical to the subject.

To provide for expression of the differentiation antigen in the chosen system, DNA encoding the differentiation antigen or a portion thereof sufficient to provide an immunologically effective expression product is inserted into a suitable expression vector. There are many vector systems known which provide for expression of incorporated genetic material in a host cell, including baculovirus vectors for use with insect cells, bacterial and yeast expression vectors, and plasmid vectors (such as psvk3) for use with mammalian cells. The use of these systems is well known in the art.

For treatment of humans with a syngeneic differentiation antigen, cDNA encoding the human differentiation antigen to be targeted must be available. cDNA is produced by reverse transcription of mRNA, and the specific cDNA encoding the PSMA antigen can be identified from a human cDNA library using probes derived from the protein sequence of the differentiation antigen, which is known in the art, examples of which are found in Seq. ID Nos. 1-2, which follow the Examples section.

Xenoexpressed PSMA antigen, like purified xenogeneic PSMA antigen, is administered to the subject individual in an amount effective to induce an immune response. The composition administered may be a lysate of cells expressing the xenoexpressed antigen, or it may be a purified or partially purified preparation of the xenoexpressed antigen.

The invention will now be further described with reference to the following, non-limiting examples:

### EXAMPLE 1

Immunization Native human PSMA was purified from an LNCaP cell lysate by immunoprecipitation using CYT-356 (Cytogen Corporation, Princeton, N.J.) and goat-anti-mouse IgG-agarose (Sigma Chemical Co., St. Louis, Mo.). The complex was washed 4 times with TNEN buffer (20 mM Tris, pH 7.5, 5 mM EDTA, 15 mM NaCl and 1% NP-40), 4 times with 0.1×TNEN+0.5 M NaCl, and once with water, after which it was resuspended in phosphate-buffered saline (PBS) and then injected intraperitoneally with Freund's adjuvant. Mice were immunized 5 times at weekly intervals.

### EXAMPLE 2

Immunization In other experiments, mice were immunized with DNA vaccines in which full-length human or mouse PSMA cDNAs were subcloned in the expression vector pcDNA3 (Invitrogen Life Technologies, Carlsbad, Calif.) or the closely related vector, pING. Plasmids were purified from bacterial lysates using Qiagen columns (Qiagen, Valencia, Calif.), and DNA was precipitated on gold, coated on plastic tubing and injected in the skin of mice.

### EXAMPLE 3

Fusion of splenocytes An immunized mouse was rested for several months, boosted once with PSMA protein eluted from anti-mouse-agarose beads and 3 days later splenocytes were fused to the myeloma Sp2/0-Ag14 using a polyethyleneglycol-based fusion protocol. Hybridoma supernatants were screened beginning 7 days after the fusion.

### EXAMPLE 4

Expression and purification of PSMA Recombinant hPSMA extracellular domain (residues 1-750) and mPSMA extracellular domain (residues 46-752) were overproduced in a baculovirus expression system using the Bac-N-Blue transfection and pBlueBacHis2 Xpress Kits (Invitrogen Life Technologies). All recombinant proteins included an epitope tag for immunostaining (Tag) and a poly-HIS tag for affinity chromatography at the amino-terminus, which were contributed by the expression vector pBlueBacHis2A.

Recombinant proteins were expressed in High Five insect cells (Invitrogen, San Diego, Calif.) grown as a monolayer. Cells were infected at a multiplicity of infection of 5:1 and harvested at 62-72 hr. The recombinant proteins were almost entirely insoluble and were isolated from inclusion bodies in 8 M urea, 150 mM NaCl and 1 mM EDTA.

A soluble form of hPSMA was also prepared for the final immunization of the C57BL/6 mouse used in the second fusion. The hPSMA extracellular domain (ECD; aa 40-750) was inserted with a GST tag and a thrombin cleavage site into the pMelBacA vector (Invitrogen Life Technologies). The protein was purified from cell culture media by ammonium sulfate precipitation, glutathione 4B column binding, thrombin cleavage and size exclusion. Purity was assessed by SDS-PAGE (>99%). hPSMA (10 mg resuspended in 50 μl saline) was injected intravenously 3 days prior to the fusion.

### EXAMPLE 5

Immunoblotting Whole cell lysates were prepared by vortexing 10′7 cultured cells/ml lysis buffer [50 mM HEPES, pH 7.5, 15 mM MgCl2, 10% glycerol, 1 mM EGTA, 2 mM Na-Vanadate, 1% TritonX-100, 0.5 μg/ml leupeptin, 10 μg/ml aprotinin, 1 μg/ml pepstatin and 100 μg/ml phenylmethylsulfonyl fluoride (PMSF)]. Prior to use in immunoblotting or in the immunoprecipitation Western blot (IP-Western) assay, lysates were precleared for 10 min by centrifugation at 14,000 rpm in a microcentrifuge at 4° C.

For immunoblotting, whole cell lysate (10 μl) was separated by SDS-PAGE on 7.5 or 10% PAGE Gold Tris-glycine gels (Bio-Whittaker, Walkersville, Md.), and transferred electrophoretically to Immobilon membranes (Millipore, Billerica, Mass.). Membranes were blocked in PBS+5% nonfat dry milk and then incubated with mouse sera, hybridoma supernatant or with anti-Tag MAb (Invitrogen Life Technologies) for 1 hr at room temperature. After 4 washes with PBS+0.1% Tween20 (PBST), a combination of horseradish peroxidase (POD)-conjugated goat-anti-mouse IgG1, IgG2a and IgG2b (Southern Biotech, Birmingham, Ala.), secondary antibodies were added for 1 hr at room temperature. After 4 washes with PBST, POD-conjugated antibodies were detected by ECL™ (Amersham Biosciences, Piscataway, N.J.) and autoradiography.

For immunoprecipitation, 50 μl LNCaP cell lysate was incubated with 50 μl hybridoma supernatant and incubated for 1 hr on ice. The immune complex was precipitated by addition of 10 μl packed volume of a goat-anti-mouse IgG-agarose bead (Sigma Chemical Co.), with a capacity of 30 μg mouse IgG. After 1 hr on ice, immune complexes were washed 4 times in cold PBS and eluted by boiling in SDS-PAGE sample buffer and the entire sample was loaded in 1 lane of a 7.5% Tris-glycine gel (BioWhittaker). After electrophoresis and transfer to Immobilon membranes, as described above, hPSMA was detected on the membrane with 6 μg/ml of a modified 7E11-05 monoclonal antibody, CYT-356 (Cytogen Corporation, Princeton, N.J.), followed by incubation with goat-anti-mouse IgG1-POD and developed with ECL.

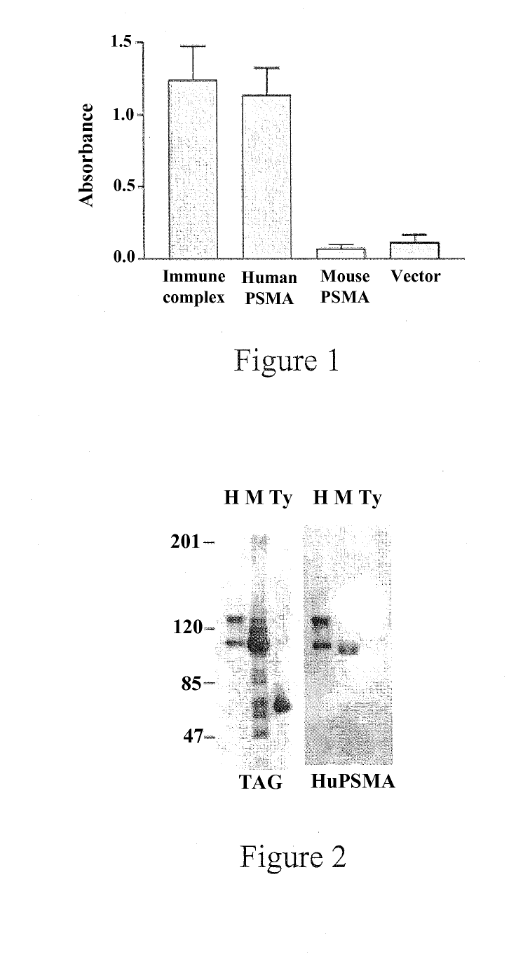

Western blots confirmed the specificity of the response to the hPSMA DNA vaccine (FIG. 2). Three recombinant proteins hPSMA (H), mPSMA (M) and human tyrosinase (Ty) were stained with the MAb anti-TAG (TAG) and with a 1/400 dilution of sera from a mouse immunized with hPSMA DNA (HuPSMA) that recognizes hPSMA and mPSMA but not Tyrosinase. The molecular weight of the recombinant mPSMA protein is less than that of hPSMA because this construct contained only the extracellular domain. No response was elicited against an unrelated recombinant glycoprotein (human tyrosinase (Ty)), which was produced in parallel in baculovirus.

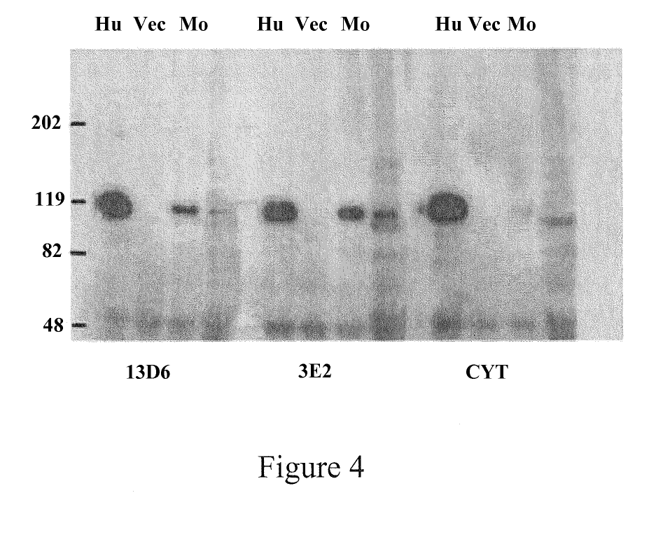

The specificity of representative clones was confirmed by Western blot analysis of lysates prepared from PSMA-positive LNCaP prostate carcinoma cells, or from NIH 3T3-hPSMA or NIH 3T3-mPSMA (FIG. 4). In FIG. 4, cross-reactive monoclonal antibodies were obtained from a mouse immunized with hPSMA protein. Whole cell lysates were prepared from NIH 3T3 cells transduced with empty SFV vector (vec), hPSMA (Hu) or mPSMA (Mo). After separation by SDS-PAGE, PSMA was visualized by incubation with cross-reactive (13D6, 3E2) or hPSMA-specific CYT-356 (CYT) monoclonal antibodies. There were no clones reactive to mPSMA alone.

### EXAMPLE 6

ELISA with hPSMA-Bac and mPSMA-Bac-coated plates Corning ELISA plates were coated with 0.2 μg recombinant protein in 8 M urea, 150 mM NaCl for at least 12 hr, rinsed twice with PBS and blocked with PBS+3% BSA for at least 1 hr. Plates were incubated with mouse sera or hybridoma supernatant and developed with a cocktail of isotype-specific goat-anti-mouse (anti-IgG3, anti-IgG1, anti-IgG2b and anti-IgG2a) alkaline phosphate-conjugated antibodies (Southern Biotech) and the substrate p-nitro-phenyl phosphate (Sigma Chemical Co.) in 10 mM diethanolamine (pH 9.5) and 0.5 mM MgCl2. Sera were diluted 1/200 for mice that received immune complexes and 1/100 for all other groups.

All mice immunized with an hPSMA DNA vaccine (10/10) or with immune complexes containing hPSMA protein (9/9) produced antibodies to both recombinant human and mouse PSMA proteins, as measured by ELISA (FIG. 1). There was no response to hPSMA or mPSMA by ELISA in mice immunized with either mPSMA DNA (9/9) or the control vector pcDNA (10/10).

Each supernatant was screened in triplicate wells coated with recombinant hPSMA, mPSMA, or human tyrosinase proteins. Supernatants that recognized either human or mouse PSMA, but not tyrosinase, were scored positive and were retested. In the primary screen, 47 of 1511 tested wells (3%) were confirmed positive (absorbance at least 5 times higher than that of human tyrosinase) and 42 clones were studied further. A high proportion of stable MAbs identified by ELISA were cross-reactive with mPSMA (36/42, or 86%). There were no clones reactive to mPSMA alone. The specificity of representative clones was confirmed by Western blot analysis of lysates prepared from PSMA-positive LNCaP prostate carcinoma cells, or from NIH 3T3-hPSMA or NIH 3T3-mPSMA. These results eliminated the possibility that the determinants seen by these antibodies are an artifact of the insect cell expression system.

### EXAMPLE 7

Flow cytometry The 3T3 cells (10̂5) were incubated with 1-5 μl sera or with 50 μl hybridoma supernatant for 1 hr on ice. Cells were washed with PBS+1% bovine serum albumin (BSA), incubated 30 min with goat-anti mouse IgG-FITC (Southern Biotech) and after washing were read in a FACSCAN cytometer (Becton Dickinson, San Jose, Calif.). In some cases, isotype-specific secondary reagents were used in place of the goat anti IgG-FITC.

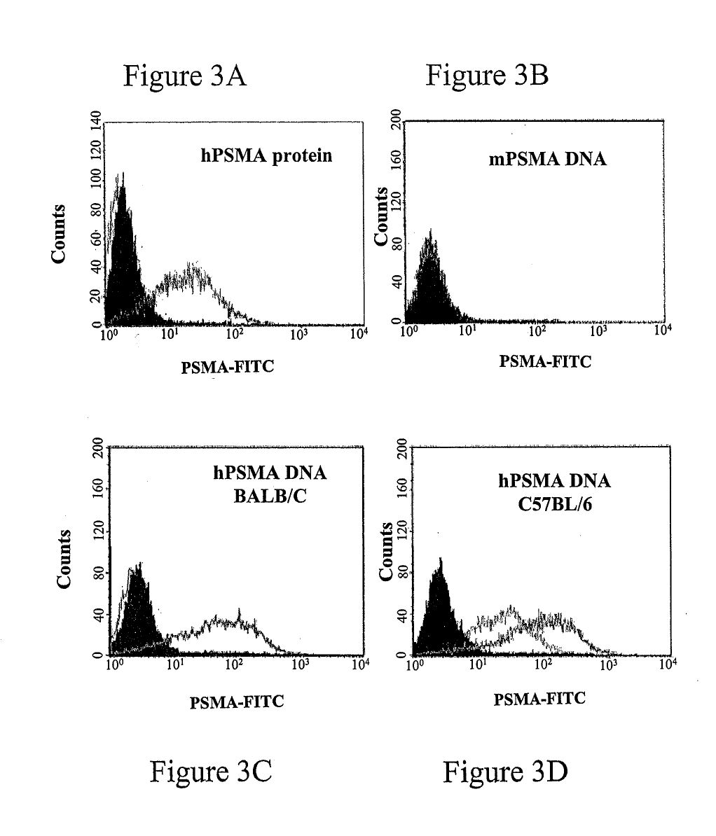

All mice immunized with an immune complex containing hPSMA produced antibodies to native hPSMA. Both BALB/C and C57BL/6 mice produced antibodies to native mPSMA after immunization with an hPSMA DNA vaccine; however, the frequencies differed. In data combined from 2 separate experiments, sera from BALB/C mice and C57BL/6 mice immunized with hPSMA DNA reacted with native cell surface mPSMA (See FIG. 3A-D). The intensity of the staining for mPSMA was typically lower then that seen for hPSMA. In all cases, there was no staining of control NIH 3T3-vector cells. C57BL/6 mice immunized with an mPSMA DNA vaccine did not produce antibodies to human or mouse PSMA (FIG. 3B).

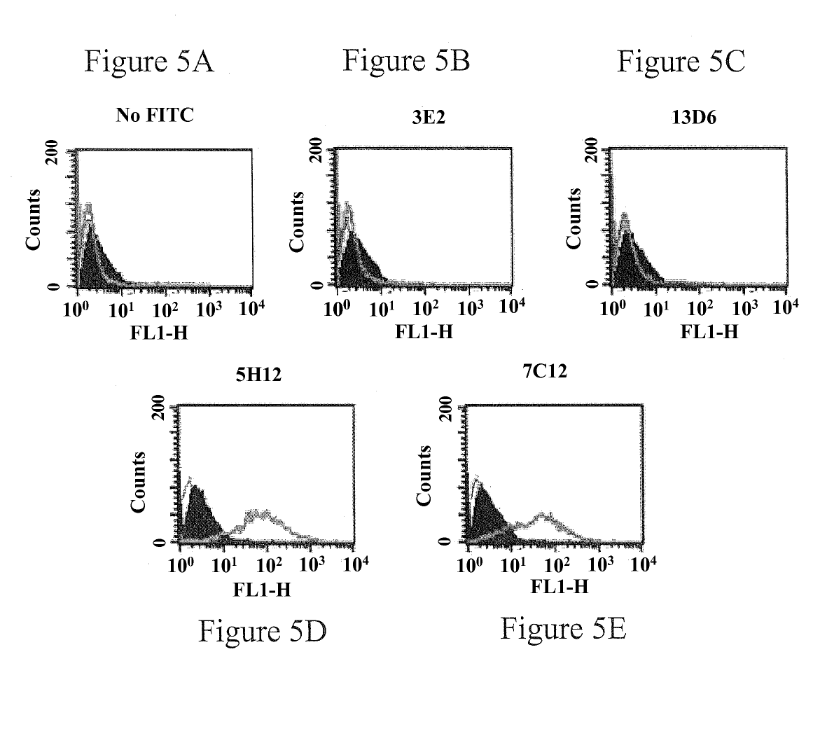

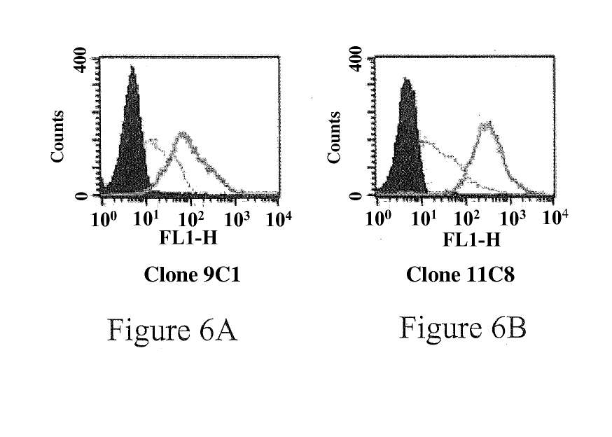

Representative supernatants were used to stain NIH 3T3 cells transduced with empty SFV vector (solid), hPSMA (dark line) or mPSMA (light line). In FIGS. 5A-E, flow cytometry data is presented for representative hybridoma supernatants isolated from a mouse immunized with hPSMA protein. As seen in FIGS. 5D-E, 5H12 and 7C12 are specific for native hPSMA. In FIGS. 6A-B, flow cytometry of cross-reactive hybridomas is presented.

### EXAMPLE 8

Immunohistochemistry Tissues were fixed in 4% paraformaldehyde, embedded in paraffin and 8 mm thick sections were prepared. The immunohistochemical detection was performed using the MOM kit (Vector Laboratories, Burlingame, Calif.). Either rabbit sera at 1/4,000 dilution or mouse monoclonal antibodies (1 μg/ml) were used. Quenching of endogenous peroxidase was performed using H₂O₂, followed by citric buffer antigen retrieval for epitope unmasking. Blocking, incubation with the biotinylated secondary antibody and the detection steps were performed as recommended by Vector Laboratories.

### EXAMPLE 9

Prime Boost To identify monoclonal antibodies to native mPSMA following vaccination with xenogeneic hPSMA-antibody immune complexes, a prime-boost strategy was performed using xenogeneic vaccination for priming followed by a final boost with xenogeneic hPSMA protein. Female BALB/C (n=15) and C57BL/6 (n=9) mice were immunized 5 times at weekly intervals with an hPSMA DNA vaccine. Sera were tested by flow cytometry, comparing the mean fluorescence intensity of control 3T3-vector cells versus 3T3-mPSMA cells stained with the same sera, under the same conditions. The best responder was identified as the animal with the highest binding to 3T3-mPSMA in the absence of binding to 3T3-vector cells. This C57BL/6 mouse was boosted intravenously with 10 μg purified recombinant hPSMA protein prior to fusion. Hybridoma supernatants were screened by flow cytometry using 3T3-mPSMA. Two positive pools, 9C1 (IgG2b) and 11C8 (IgG2b), were identified among 2,082 wells, which contained approximately 4,000 hybridomas. Hybridomas specific for mPSMA were cloned from each of the positive wells, and both of these pools were found to be specific for native mouse and human PSMA, with no recognition of 3T3-vector cells.

To compare the number of wells specific for human and mouse PSMA, 284 representative wells from the fusion were also tested by flow cytometry for recognition of native hPSMA using 3T3-hPSMA. In this screen, we identified 3 positive samples out of 284 (1%). Normalized to the entire fusion, we estimate that 22 wells out of 2,082 recognized native hPSMA, and 2 of these (10%) cross-reacted with mPSMA.

### SEQUENCES

The sequences for the coding sequence of two isoforms of Homo sapiens PSMA are known in the art. The cDNA of human PSMA was sequenced in Israeli, R. S., Powell, C. T., Fair, W. R. and Heston, W. D., Molecular cloning of a complementary DNA encoding a prostate-specific membrane antigen, 53 Cancer Res. 227-230 (1993).

| Seq. ID No. 1-human PSMA (isofom 1) | |
|---|---|
| 1 | atgtggaatc tccttcacga aaccgactcg gctgtggcca ccgcgcgccg cccgcgctgg |
| 61 | ctgtgcgctg gggcgctggt gctggcgggt ggcttctttc tcctcggctt cctcttcggg |
| 121 | tggtttataa aatcctccaa tgaagctact aacattactc caaagcataa tatgaaagca |
| 181 | tttttggatg aattgaaagc tgagaacatc aagaagttct tatataattt tacacagata |
| 241 | ccacatttag caggaacaga acaaaacttt cagcttgcaa agcaaattca atcccagtgg |
| 301 | aaagaatttg gcctggattc tgttgagcta gcacattatg atgtcctgtt gtcctaccca |
| 361 | aataagactc atcccaacta catctcaata attaatgaag atggaaatga gattttcaac |
| 421 | acatcattat ttgaaccacc tcctccagga tatgaaaatg tttcggatat tgtaccacct |
| 481 | ttcagtgctt tctctcctca aggaatgcca gagggcgatc tagtgtatgt taactatgca |
| 541 | cgaactgaag acttctttaa attggaacgg gacatgaaaa tcaattgctc tgggaaaatt |
| 601 | gtaattgcca gatatgggaa agttttcaga ggaaataagg ttaaaaatgc ccagctggca |
| 661 | ggggccaaag gagtcattct ctactccgac cctgctgact actttgctcc tggggtgaag |
| 721 | tcctatccag atggttggaa tcttcctgga ggtggtgtcc agcgtggaaa tatcctaaat |
| 781 | ctgaatggtg caggagaccc tctcacacca ggttacccag caaatgaata tgcttatagg |
| 841 | cgtggaattg cagaggctgt tggtcttcca agtattcctg ttcatccaat tggatactat |
| 901 | gatgcacaga agctcctaga aaaaatgggt ggctcagcac caccagatag cagctggaga |
| 961 | ggaagtctca aagtgcccta caatgttgga cctggcttta ctggaaactt ttctacacaa |
| 1021 | aaagtcaaga tgcacatcca ctctaccaat gaagtgacaa gaatttacaa tgtgataggt |
| 1081 | actctcagag gagcagtgga accagacaga tatgtcattc tgggaggtca ccgggactca |
| 1141 | tgggtgtttg gtggtattga ccctcagagt ggagcagctg ttgttcatga aattgtgagg |
| 1201 | agctttggaa cactgaaaaa ggaagggtgg agacctagaa gaacaatttt gtttgcaagc |
| 1261 | tgggatgcag aagaatttgg tcttcttggt tctactgagt gggcagagga gaattcaaga |
| 1321 | ctccttcaag agcgtggcgt ggcttatatt aatgctgact catctataga aggaaactac |
| 1381 | actctgagag ttgattgtac accgctgatg tacagcttgg tacacaacct aacaaaagag |
| 1441 | ctgaaaagcc ctgatgaagg ctttgaaggc aaatctcttt atgaaagttg gactaaaaaa |
| 1501 | agtccttccc cagagttcag tggcatgccc aggataagca aattgggatc tggaaatgat |
| 1561 | tttgaggtgt tcttccaacg acttggaatt gcttcaggca gagcacggta tactaaaaat |
| 1621 | tgggaaacaa acaaattcag cggctatcca ctgtatcaca gtgtctatga aacatatgag |
| 1681 | ttggtggaaa agttttatga tccaatgttt aaatatcacc tcactgtggc ccaggttcga |
| 1741 | ggagggatgg tgtttgagct agccaattcc atagtgctcc cttttgattg tcgagattat |
| 1801 | gctgtagttt taagaaagta tgctgacaaa atctacagta tttctatgaa acatccacag |
| 1861 | gaaatgaaga catacagtgt atcatttgat tcactttttt ctgcagtaaa gaattttaca |
| 1921 | gaaattgctt ccaagttcag tgagagactc caggactttg acaaaagcaa cccaatagta |
| 1981 | ttaagaatga tgaatgatca actcatgttt ctggaaagag catttattga tccattaggg |
| 2041 | ttaccagaca ggccttttta taggcatgtc atctatgctc caagcagcca caacaagtat |
| 2101 | gcaggggagt cattcccagg aatttatgat gctctgtttg atattgaaag caaagtggac |
| 2161 | ccttccaagg cctggggaga agtgaagaga cagatttatg ttgcagcctt cacagtgcag |
| 2221 | gcagctgcag agactttgag tgaagtagcc taa |
| Seq. ID No. 2-Human PSMA (isoform 2) | |
| 1 | atgtggaatc tccttcacga aaccgactcg gctgtggcca ccgcgcgccg cccgcgctgg |
| 61 | ctgtgcgctg gggcgctggt gctggcgggt ggcttctttc tcctcggctt cctcttcggg |
| 121 | tggtttataa aatcctccaa tgaagctact aacattactc caaagcataa tatgaaagca |
| 181 | tttttggatg aattgaaagc tgagaacatc aagaagttct tatataattt tacacagata |
| 241 | ccacatttag caggaacaga acaaaacttt cagcttgcaa agcaaattca atcccagtgg |
| 301 | aaagaatttg gcctggattc tgttgagcta gcacattatg atgtcctgtt gtcctaccca |
| 361 | aataagactc atcccaacta catctcaata attaatgaag atggaaatga gattttcaac |
| 421 | acatcattat ttgaaccacc tcctccagga tatgaaaatg tttcggatat tgtaccacct |
| 481 | ttcagtgctt tctctcctca aggaatgcca gagggcgatc tagtgtatgt taactatgca |
| 541 | cgaactgaag acttctttaa attggaacgg gacatgaaaa tcaattgctc tgggaaaatt |
| 601 | gtaattgcca gatatgggaa agttttcaga ggaaataagg ttaaaaatgc ccagctggca |
| 661 | ggggccaaag gagtcattct ctactccgac cctgctgact actttgctcc tggggtgaag |
| 721 | tcctatccag atggttggaa tcttcctgga ggtggtgtcc agcgtggaaa tatcctaaat |
| 781 | ctgaatggtg caggagaccc tctcacacca ggttacccag caaatgaata tgcttatagg |
| 841 | cgtggaattg cagaggctgt tggtcttcca agtattcctg ttcatccaat tggatactat |
| 901 | gatgcacaga agctcctaga aaaaatgggt ggctcagcac caccagatag cagctggaga |
| 961 | ggaagtctca aagtgcccta caatgttgga cctggcttta ctggaaactt ttctacacaa |
| 1021 | aaagtcaaga tgcacatcca ctctaccaat gaagtgacaa gaatttacaa tgtgataggt |
| 1081 | actctcagag gagcagtgga accagacaga tatgtcattc tgggaggtca ccgggactca |
| 1141 | tgggtgtttg gtggtattga ccctcagagt ggagcagctg ttgttcatga aattgtgagg |
| 1201 | agctttggaa cactgaaaaa ggaagggtgg agacctagaa gaacaatttt gtttgcaagc |
| 1261 | tgggatgcag aagaatttgg tcttcttggt tctactgagt gggcagagga gaattcaaga |
| 1321 | ctccttcaag agcgtggcgt ggcttatatt aatgctgact catctataga aggaaactac |
| 1381 | actctgagag ttgattgtac accgctgatg tacagcttgg tacacaacct aacaaaagag |
| 1441 | ctgaaaagcc ctgatgaagg ctttgaaggc aaatctcttt atgaaagttg gactaaaaaa |
| 1501 | agtccttccc cagagttcag tggcatgccc aggataagca aattgggatc tggaaatgat |
| 1561 | tttgaggtgt tcttccaacg acttggaatt gcttcaggca gagcacggta tactaaaaat |
| 1621 | tgggaaacaa acaaattcag cggctatcca ctgtatcaca gtgtctatga aacatatgag |
| 1681 | ttggtggaaa agttttatga tccaatgttt aaatatcacc tcactgtggc ccaggttcga |
| 1741 | ggagggatgg tgtttgagct agccaattcc atagtgctcc cttttgattg tcgagattat |
| 1801 | gctgtagttt taagaaagta tgctgacaaa atctacagta tttctatgaa acatccacag |
| 1861 | gaaatgaaga catacagtgt atcatttgat tcactttttt ctgcagtaaa gaattttaca |
| 1921 | gaaattgctt ccaagttcag tgagagactc caggactttg acaaaagcaa gcatgtcatc |
| 1981 | tatgctccaa gcagccacaa caagtatgca ggggagtcat tcccaggaat ttatgatgct |
| 2041 | ctgtttgata ttgaaagcaa agtggaccct tccaaggcct ggggagaagt gaagagacag |
| 2101 | atttatgttg cagccttcac agtgcaggca gctgcagaga ctttgagtga agtagcctaa |
| Seq. ID No. 3-mouse PSMA (isoform 1) | |
| 1 | atgtggaacg cactgcagga cagagactcc gcggaggtcc tgggacaccg ccagcgctgg |
| 61 | ctccgtgttg ggacactggt gctggcttta accggaacct tcctcattgg cttcctcttt |
| 121 | gggtggttta taaaaccttc caatgaagct actggtaatg tttcccattc tggcatgaag |
| 181 | aaggagtttt tgcatgaatt gaaggctgag aacatcaaaa aatttttata caatttcaca |
| 241 | cggacaccac acttggcagg aacacaaaat aattttgagc ttgcaaagca aattcatgac |
| 301 | cagtggaaag aatttggcct ggatttggtt gagttatccc attacgatgt cttgctgtcc |
| 361 | tatccaaata aaactcatcc taactatatc tcaataatta atgaagatgg aaatgagatt |
| 421 | ttcaaaacat cattatctga acagccaccc ccaggatatg agaatatatc agatgtagtg |
| 481 | ccaccataca gtgccttctc tccacaaggg acaccagagg gtgatctagt gtatgtcaac |
| 541 | tatgcacgaa ctgaagactt ctttaaactg gaacgggaaa tgaagatcag ttgttctggg |
| 601 | aagattgtga ttgccagata tgggaaagtg ttcagaggaa atatggttaa aaatgctcaa |
| 661 | ctggcagggg caaaaggaat gattctgtac tcagaccctg ctgactactt tgttcctgcg |
| 721 | gtgaagtcct atccagatgg ctggaacctc cctggaggtg gtgtccaacg tggaaatgtc |
| 781 | ttaaatctta atggtgcagg tgacccgctc acaccaggtt acccagcaaa tgaacatgct |
| 841 | tataggcatg agttgacaaa cgctgttggc cttccaagta ttcctgtcca tcctattgga |
| 901 | tatgatgatg cacagaaact cttagaacac atgggtggtc cagcaccccc tgacagtagc |
| 961 | tggaagggag gattaaaagt gccttacaac gtgggacctg gctttgctgg aaacttttca |
| 1021 | acacaaaagg tcaagatgca tattcactct tacactaaag tgacaagaat ctataatgtc |
| 1081 | attggcaccc tcaaaggagc tctggaacca gacagatatg ttattcttgg aggtcaccga |
| 1141 | gatgcttggg tatttggtgg cattgaccct cagagtggag cagctgttgt tcatgaaatt |
| 1201 | gtgcggagct ttggaaccct gaagaagaaa ggacggaggc ctagaaggac aattttgttt |
| 1261 | gcaagctggg atgcagaaga atttggcctt cttggttcta ctgagtgggc agaggaacat |
| 1321 | tcaagactcc tacaagagcg aggtgtggct tatattaatg ctgattcttc catagaagga |
| 1381 | aattacactc taagagttga ttgcacacca ctgatgtaca gcttagtgta caacctaaca |
| 1441 | aaagagctgc aaagcccaga tgaaggtttt gaaggaaaat ctctttatga cagctggaaa |
| 1501 | gaaaagagtc cttcacctga gttcattgga atgcccagaa ttagcaagct ggggtctggc |
| 1561 | aatgattttg aagtgttctt ccaaagactt ggaattgctt caggcagagc ccgatatact |
| 1621 | aaaaattgga aaactaacaa agtcagcagc tatcctctct atcacagtgt ctatgaaaca |
| 1681 | tatgagctgg tagtaaaatt ttatgaccca acatttaaat accacctcac tgtggcccag |
| 1741 | gttcgaggag cgatggtatt tgaacttgcc aattctatag tgcttccctt tgactgccaa |
| 1801 | agttatgctg tagctctgaa gaagtatgct gacactatct acaatatttc aatgaaacat |
| 1861 | ccacaagaaa tgaaggctta catgatatca tttgattcac tgttttctgc agtcaataat |
| 1921 | tttacagatg ttgcatctaa gttcaatcag agactgcaag agttagacaa aagcaacccc |
| 1981 | atattactga gaattatgaa tgaccagctg atgtatctgg aacgtgcatt cattgatcct |
| 2041 | ttaggcttac caggaaggcc tttctacagg catatcatct atgctccaag cagccacaac |
| 2101 | aagtatgcag gagaatcatt ccctgggatt tatgatgccc tttttgatat aagtagcaaa |
| 2161 | gtcaatgctt ctaaggcctg gaacgaagtg aagagacaga tttctattgc aacctttaca |
| 2221 | gtgcaagctg cagcagagac tctgagggaa gtagcttaa |
| Seq. ID No. 4-mouse PSMA (isoform 2) | |
| 1 | atgtggaacg cactgcagga cagagactcc gcggaggtcc tgggacaccg ccagcgctgg |
| 61 | ctccgtgttg ggacactggt gctggcttta accggaacct tcctcattgg cttcctcttt |
| 121 | gggtggttta taaaaccttc caatgaagct actggtaatg tttcccattc tggcatgaag |
| 181 | aaggagtttt tgcatgaatt gaaggctgag aacatcaaaa aatttttata caatttcaca |
| 241 | cggacaccac acttggcagg aacacaaaat aattttgagc ttgcaaagca aattcatgac |
| 301 | cagtggaaag aatttggcct ggatttggtt gagttatccc attacgatgt cttgctgtcc |
| 361 | tatccaaata aaactcatcc taactatatc tcaataatta atgaagatgg aaatgagatt |
| 421 | ttcaaaacat cattatctga acagccaccc ccaggatatg agaatatatc agatgtagtg |
| 481 | ccaccataca gtgccttctc tccacaaggg acaccagagg gtgatctagt gtatgtcaac |
| 541 | tatgcacgaa ctgaagactt ctttaaactg gaacgggaaa tgaagatcag ttgttctggg |
| 601 | aagattgtga ttgccagata tgggaaagtg ttcagaggaa atatggttaa aaatgctcaa |
| 661 | ctggcagggg caaaaggaat gattctgtac tcagaccctg ctgactactt tgttcctgcg |
| 721 | gtgaagtcct atccagatgg ctggaacctc cctggaggtg gtgtccaacg tggaaatgtc |
| 781 | ttaaatctta atggtgcagg tgacccgctc acaccaggtt acccagcaaa tgaacatgct |
| 841 | tataggcatg agttgacaaa cgctgttggc cttccaagta ttcctgtcca tcctattgga |
| 901 | tatgatgatg cacagaaact cttagaaaag gtcaagatgc atattcactc ttacactaaa |
| 961 | gtgacaagaa tctataatgt cattggcacc ctcaaaggag ctctggaacc agacagatat |
| 1021 | gttattcttg gaggtcaccg agatgcttgg gtatttggtg gcattgaccc tcagagtgga |
| 1081 | gcagctgttg ttcatgaaat tgtgcggagc tttggaaccc tgaagaagaa aggacggagg |
| 1141 | cctagaagga caattttgtt tgcaagctgg gatgcagaag aatttggcct tcttggttct |
| 1201 | actgagtggg cagaggaaca ttcaagactc ctacaagagc gaggtgtggc ttatattaat |
| 1261 | gctgattctt ccatagaagg aaattacact ctaagagttg attgcacacc actgatgtac |
| 1321 | agcttagtgt acaacctaac aaaagagctg caaagcccag atgaaggttt tgaaggaaaa |
| 1381 | tctctttatg acagctggaa agaaaagagt ccttcacctg agttcattgg aatgcccaga |
| 1441 | attagcaagc tggggtctgg caatgatttt gaagtgttct tccaaagact tggaattgct |
| 1501 | tcaggcagag cccgatatac taaaaattgg aaaactaaca aagtcagcag ctatcctctc |
| 1561 | tatcacagtg tctatgaaac atatgagctg gtagtaaaat tttatgaccc aacatttaaa |
| 1621 | taccacctca ctgtggccca ggttcgagga gcgatggtat ttgaacttgc caattctata |
| 1681 | gtgcttccct ttgactgcca aagttatgct gtagctctga agaagtatgc tgacactatc |
| 1741 | tacaatattt caatgaaaca tccacaagaa atgaaggctt acatgatatc atttgattca |
| 1801 | ctgttttctg cagtcaataa ttttacagat gttgcatcta agttcaatca gagactgcaa |
| 1861 | gagttagaca aaagcaaccc catattactg agaattatga atgaccagct gatgtatctg |
| 1921 | gaacgtgcat tcattgatcc tttaggctta ccaggaaggc ctttctacag gcatatcatc |
| 1981 | tatgctccaa gcagccacaa caagtatgca ggagaatcat tccctgggat ttatgatgcc |
| 2041 | ctttttgata taagtagcaa agtcaatgct tctaaggcct ggaacgaagt gaagagacag |
| 2101 | atttctattg caacctttac agtgcaagct gcagcagaga ctctgaggga agtagcttaa |

All references cited herein are incorporated by reference.

We claim:

1. A method for stimulating an immune response to a tissue expressing prostate specific membrane antibody (PSMA) in a subject individual of a first species, comprising administering to the subject individual an immunologically-effective amount of xenogeneic or xenoexpressed PSMA antigen.

2. The method according to claim 1, wherein the subject individual of the first species is human.

3. The method of claim 1, wherein the PSMA antigen is a xenogeneic PSMA antigen derived from a source selected from the group consisting of rodents, dogs, cats, cows, and sheep PSMA antigen.

4. The method of claim 1, wherein the step of administering is achieved by immunization with DNA encoding a xenogeneic PSMA antigen.

5. The method of claim 4, wherein the DNA immunization is achieved by immunization with liposomes comprising DNA encoding the xenogeneic PSMA antigen.

6. The method of claim 4, wherein the DNA immunization is achieved by immunization with gold particles coated with DNA encoding the xenogeneic PSMA antigen.

7. The method of claim 4, wherein the DNA encoding the PSMA antigen is an expression vector encoding the PSMA antigen.

8. The method of claim 1 wherein the immune response is a cellular or humoral response.

9. The method of claim 8 wherein the amount of xenogeneic or xenoexpressed PSMA antigen is sufficient to provide immunoprotection against growth of tumors expressing PSMA.

10. The method of claim 1 wherein the amount of xenogeneic or xenoexpressed PSMA antigen is sufficient to provide immunoprotection against growth of tumors expressing PSMA.

11. The method of claim 1, wherein the step of administering is achieved by immunization with a construct comprising DNA encoding a xenogeneic PSMA antigen, said construct resulting in expression of the xenogeneic PSMA antigen in the subject individual.

12. The method of claim 1, wherein the subject individual has prostate cancer.

13. The method of claim 12, wherein the step of administering is achieved by immunization with a construct comprising DNA encoding a xenogeneic PSMA antigen, said construct resulting in expression of the xenogeneic PSMA antigen in the subject individual.


SEQUENCE LISTING (4 sequences)

SEQ ID NO: 1 - DNA, 2143 bp, Homo sapiens
agtgcctttggttgctggagggaagaacacaatggatctggtgctaaaaagatgccttcttcatttggctgtgataggtgctttgctggctgtgggggctacaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatgggagccaggtgtggggaggacagccagtgtatccccaggaaactgacgatgcctgcatcttccctgatggtggaccttgcccatctggctcttggtctcagaagagaagctttgtttatgtctggaagacctggggccaatactggcaagttctagggggcccagtgtctgggctgagcattgggacaggcagggcaatgctgggcacacacaccatggaagtgactgtctaccatcgccggggatcccggagctatgtgcctcttgctcattccagctcagccttcaccattactgaccaggtgcctttctccgtgagcgtgtcccagttgcgggccttggatggagggaacaagcacttcctgagaaatcagcctctgacctttgccctccagctccatgaccccagtggctatctggctgaagctgacctctcctacacctgggactttggagacagtagtggaaccctgatctctcgggcacttgtggtcactcatacttacctggagcctggcccagtcactgcccaggtggtcctgcaggctgccattcctctcacctcctgtggctcctccccagttccaggcaccacagatgggcacaggccaactgcagaggcccctaacaccacagctggccaagtgcctactacagaagttgtgggtactacacctggtcaggcgccaactgcagagccctctggaaccacatctgtgcaggtgccaaccactgaagtcataagcactgcacctgtgcagatgccaactgcagagagcacaggtatgacacctgagaaggtgccagtttcagaggtcatgggtaccacactggcagagatgtcaactccagaggctacaggtatgacacctgcagaggtatcaattgtggtgctttctggaaccacagctgcacaggtaacaactacagagtgggtggagaccacagctagagagctacctatccctgagcctgaaggtccagatgccagctcaatcatgtctacggaaagtattacaggttccctgggccccctgctggatggtacagccaccttaaggctggtgaagagacaagtccccctggattgtgttctgtatcgatatggttccttttccgtcaccctggacattgtccagggtattgaaagtgccgagatcctgcaggctgtgccgtccggtgagggggatgcatttgagctgactgtgtcctgccaaggcgggctgcccaaggaagcctgcatggagatctcatcgccagggtgccagccccctgcccagcggctgtgccagcctgtgctacccagcccagcctgccagctggttctgcaccagatactgaagggtggctcggggacatactgcctcaatgtgtctctggctgataccaacagcctggcagtggtcagcacccagcttatcatgcctggtcaagaagcaggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaatagccccctcctcagtgggcagcaggtctgagtactctcatatgatgctgtgattttcctggagttgacagaaacacctatatttcccccagtcttccctgggagactactattaactgaaataaatactcagagcctgaaaaaaaaaaaaaaaaaa

SEQ ID NO: 2 - DNA, 2160 bp, Homo sapiens
atgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaagcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaa

SEQ ID NO: 3 - DNA, 2259 bp, Mus musculus
atgtggaacgcactgcaggacagagactccgcggaggtcctgggacaccgccagcgctggctccgtgttgggacactggtgctggctttaaccggaaccttcctcattggcttcctctttgggtggtttataaaaccttccaatgaagctactggtaatgtttcccattctggcatgaagaaggagtttttgcatgaattgaaggctgagaacatcaaaaaatttttatacaatttcacacggacaccacacttggcaggaacacaaaataattttgagcttgcaaagcaaattcatgaccagtggaaagaatttggcctggatttggttgagttatcccattacgatgtcttgctgtcctatccaaataaaactcatcctaactatatctcaataattaatgaagatggaaatgagattttcaaaacatcattatctgaacagccacccccaggatatgagaatatatcagatgtagtgccaccatacagtgccttctctccacaagggacaccagagggtgatctagtgtatgtcaactatgcacgaactgaagacttctttaaactggaacgggaaatgaagatcagttgttctgggaagattgtgattgccagatatgggaaagtgttcagaggaaatatggttaaaaatgctcaactggcaggggcaaaaggaatgattctgtactcagaccctgctgactactttgttcctgcggtgaagtcctatccagatggctggaacctccctggaggtggtgtccaacgtggaaatgtcttaaatcttaatggtgcaggtgacccgctcacaccaggttacccagcaaatgaacatgcttataggcatgagttgacaaacgctgttggccttccaagtattcctgtccatcctattggatatgatgatgcacagaaactcttagaacacatgggtggtccagcaccccctgacagtagctggaagggaggattaaaagtgccttacaacgtgggacctggctttgctggaaacttttcaacacaaaaggtcaagatgcatattcactcttacactaaagtgacaagaatctataatgtcattggcaccctcaaaggagctctggaaccagacagatatgttattcttggaggtcaccgagatgcttgggtatttggtggcattgaccctcagagtggagcagctgttgttcatgaaattgtgcggagctttggaaccctgaagaagaaaggacggaggcctagaaggacaattttgtttgcaagctgggatgcagaagaatttggccttcttggttctactgagtgggcagaggaacattcaagactcctacaagagcgaggtgtggcttatattaatgctgattcttccatagaaggaaattacactctaagagttgattgcacaccactgatgtacagcttagtgtacaacctaacaaaagagctgcaaagcccagatgaaggttttgaaggaaaatctctttatgacagctggaaagaaaagagtccttcacctgagttcattggaatgcccagaattagcaagctggggtctggcaatgattttgaagtgttcttccaaagacttggaattgcttcaggcagagcccgatatactaaaaattggaaaactaacaaagtcagcagctatcctctctatcacagtgtctatgaaacatatgagctggtagtaaaattttatgacccaacatttaaataccacctcactgtggcccaggttcgaggagcgatggtatttgaacttgccaattctatagtgcttccctttgactgccaaagttatgctgtagctctgaagaagtatgctgacactatctacaatatttcaatgaaacatccacaagaaatgaaggcttacatgatatcatttgattcactgttttctgcagtcaataattttacagatgttgcatctaagttcaatcagagactgcaagagttagacaaaagcaaccccatattactgagaattatgaatgaccagctgatgtatctggaacgtgcattcattgatcctttaggcttaccaggaaggcctttctacaggcatatcatctatgctccaagcagccacaacaagtatgcaggagaatcattccctgggatttatgatgccctttttgatataagtagcaaagtcaatgcttctaaggcctggaacgaagtgaagagacagatttctattgcaacctttacagtgcaagctgcagcagagactctgagggaagtagcttaa

SEQ ID NO: 4 - DNA, 2160 bp, Mus musculus
atgtggaacgcactgcaggacagagactccgcggaggtcctgggacaccgccagcgctggctccgtgttgggacactggtgctggctttaaccggaaccttcctcattggcttcctctttgggtggtttataaaaccttccaatgaagctactggtaatgtttcccattctggcatgaagaaggagtttttgcatgaattgaaggctgagaacatcaaaaaatttttatacaatttcacacggacaccacacttggcaggaacacaaaataattttgagcttgcaaagcaaattcatgaccagtggaaagaatttggcctggatttggttgagttatcccattacgatgtcttgctgtcctatccaaataaaactcatcctaactatatctcaataattaatgaagatggaaatgagattttcaaaacatcattatctgaacagccacccccaggatatgagaatatatcagatgtagtgccaccatacagtgccttctctccacaagggacaccagagggtgatctagtgtatgtcaactatgcacgaactgaagacttctttaaactggaacgggaaatgaagatcagttgttctgggaagattgtgattgccagatatgggaaagtgttcagaggaaatatggttaaaaatgctcaactggcaggggcaaaaggaatgattctgtactcagaccctgctgactactttgttcctgcggtgaagtcctatccagatggctggaacctccctggaggtggtgtccaacgtggaaatgtcttaaatcttaatggtgcaggtgacccgctcacaccaggttacccagcaaatgaacatgcttataggcatgagttgacaaacgctgttggccttccaagtattcctgtccatcctattggatatgatgatgcacagaaactcttagaaaaggtcaagatgcatattcactcttacactaaagtgacaagaatctataatgtcattggcaccctcaaaggagctctggaaccagacagatatgttattcttggaggtcaccgagatgcttgggtatttggtggcattgaccctcagagtggagcagctgttgttcatgaaattgtgcggagctttggaaccctgaagaagaaaggacggaggcctagaaggacaattttgtttgcaagctgggatgcagaagaatttggccttcttggttctactgagtgggcagaggaacattcaagactcctacaagagcgaggtgtggcttatattaatgctgattcttccatagaaggaaattacactctaagagttgattgcacaccactgatgtacagcttagtgtacaacctaacaaaagagctgcaaagcccagatgaaggttttgaaggaaaatctctttatgacagctggaaagaaaagagtccttcacctgagttcattggaatgcccagaattagcaagctggggtctggcaatgattttgaagtgttcttccaaagacttggaattgcttcaggcagagcccgatatactaaaaattggaaaactaacaaagtcagcagctatcctctctatcacagtgtctatgaaacatatgagctggtagtaaaattttatgacccaacatttaaataccacctcactgtggcccaggttcgaggagcgatggtatttgaacttgccaattctatagtgcttccctttgactgccaaagttatgctgtagctctgaagaagtatgctgacactatctacaatatttcaatgaaacatccacaagaaatgaaggcttacatgatatcatttgattcactgttttctgcagtcaataattttacagatgttgcatctaagttcaatcagagactgcaagagttagacaaaagcaaccccatattactgagaattatgaatgaccagctgatgtatctggaacgtgcattcattgatcctttaggcttaccaggaaggcctttctacaggcatatcatctatgctccaagcagccacaacaagtatgcaggagaatcattccctgggatttatgatgccctttttgatataagtagcaaagtcaatgcttctaaggcctggaacgaagtgaagagacagatttctattgcaacctttacagtgcaagctgcagcagagactctgagggaagtagcttaa